(19) United States
(12) Patent Application Publication
(10) Pub. No.: US 2011/0125411 A1
(43) Pub. Date: May 26, 2011

(54) Uniquemer Algorithm for Identification of Conserved and Unique Subsequences

(75) Inventors: Shea N. Gardner, Oakland, CA (US); Thomas A. Kuczmarski, Madison, WI (US)

(73) Assignee: LAWRENCE LIVERMORE NATIONAL SECURITY, LLC, Livermore, CA (US)

(21) Appl. No.: 12/051,766

(22) Filed: Mar. 19, 2008

(51) Int. Cl. G06F 19/00; G01N 33/48

(52) U.S. Cl. 702/20

(57) ABSTRACT

A first protein sequence associated with the organism is identified, wherein the first protein sequence comprises a plurality of ordered residues. A plurality of sub-sequences is generated based on the first protein sequence, wherein each sub-sequence comprises a plurality of contiguous residues and a starting residue number of each sub-sequence differs from a starting residue number of another sub-sequence by one position in the first protein sequence. A first unique sub-sequence comprising a first set of contiguous residues based on the plurality of sub-sequences is identified, wherein the first unique sub-sequence is specific to the organism and is identified based on a dataset of protein sequences and stored.

### STATEMENT REGARDING FEDERALLY FUNDED RESEARCH

This invention was made in the course of or under prime Contract No. DE-AC52-07NA27344 between the U.S. Department of Energy and Lawrence Livermore National Security, LLC. This Record of Invention is prepared for the Office of the Assistant General Counsel for Patents, U.S. Department of Energy.

### BACKGROUND OF THE INVENTION

1. Field of Invention

The present invention relates to the field of bioinformatics. More specifically, the invention relates to computational methods of identifying protein signatures to uniquely identify an organism.

### 2. Background of the Invention

A motif or signature is a defined region on a target protein that may be used to specifically identify that protein or, indirectly, the organism that produces it. There is an increased need to rapidly develop highly specific detection assays for organisms which cause biological threat. The identification of signatures specific to organisms of interest such as pathogens or toxins allows the rapid development of detection assays.

Non-computational methods of identifying protein signatures for high-affinity ligand-based detection include generation of antibodies to whole organisms, whole proteins or peptides. Non-computational methods of identifying protein signatures for reagent development include screening of compounds. In addition to being costly and time-consuming, non-computational methods are based on the principle of discovery and provide no a priori quantitative characterization of the protein residues forming the signature. Consequently, traditional methods based on, e.g., antibody generation or compound library screens provide little information that can be used for down-selecting or targeting the possible pool of reagents. In addition, if an antibody binds to a protein, it is possible that only a subset of residues within the protein bind the antibody, and further experimentation is required to find the residues responsible for antibody binding.

Current computational methods for identifying protein signatures are largely based on the analysis of conservation through multiple sequence alignment. Residue conservation is an indirect measure of functional or structural importance. Sequence alignments are carried out using utilities such as, e.g., BLAST (available from the National Center for Biotechnology Information website). From such sequence alignments, residues that are conserved within a set of proteins can be identified. Despite the power of techniques which use conservation for generating protein signatures or motifs, they suffer from several shortcomings.

Although signatures based on conservation can often indicate areas that are functionally or structurally important, such signatures are not always specific to a protein of interest. For example, residues found in functional domains such as the basic leucine zipper domain are conserved. However, basic leucine zipper domains are found in large number of proteins and therefore cannot be used to generate a signature which specifically identifies a given protein. Also, methods based on conservation require the a priori knowledge of a group of close homologs or proteins, information which often is unavailable. Further, residues that are conserved in a protein from one organism are also conserved in their homologs and by definition not unique to an organism of interest. Similarly, residues that are conserved within a group of proteins structures with different functional characteristics are not unique to a set of proteins with the same functional characteristic.

Accordingly, improved methods of identifying protein signatures that are unique to an organism are needed.

### SUMMARY OF THE INVENTION

The above and other needs are met by systems and computer program products for identifying a sub-sequence that is unique to an organism

One aspect provides a method of identifying a sub-sequence that is unique to an organism. A first protein sequence associated with the organism is identified, wherein the first protein sequence comprises a plurality of ordered residues. A plurality of sub-sequences is generated based on the first protein sequence, wherein each sub-sequence comprises a plurality of contiguous residues and a starting residue number of each sub-sequence differs from a starting residue number of another sub-sequence by one position in the first protein sequence. A first unique sub-sequence comprising a first set of contiguous residues based on the plurality of sub-sequences is identified, wherein the first unique sub-sequence is specific to the organism and is identified based on a dataset of protein sequences and stored.

Another aspect provides a method of identifying a sub-sequence that is unique to an organism. A first protein sequence associated with the organism is identified, wherein the first protein sequence comprises a plurality of ordered residues. A plurality of sub-sequences is generated based on the first protein sequence, wherein each sub-sequence comprises a plurality of contiguous residues ranging from 4-10 residues in length. A first unique sub-sequence comprising a first set of contiguous residues is identified based on the plurality of sub-sequences, wherein the first unique sub-sequence is specific to the organism and is identified based on a dataset of protein sequences and stored.

Other aspects are embodied as a computer-readable storage medium encoded with computer program code for identifying a sub-sequence that is unique to an organism according to the methods described above.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter, which is defined solely by the claims.

### BRIEF DESCRIPTION OF THE DRAWINGS

FIG. 1 is a high-level block diagram of a computing environment 100 according to one embodiment.

FIG. 2 is a high-level block diagram illustrating a detailed view of a Uniquemer Engine 110 according to one embodiment.

FIG. 3 provides a conceptual illustration of the uniquemer algorithm.

FIG. 4 is a flowchart illustrating steps performed by the Uniquemer Engine 110 to determine uniquemers contained in a protein sequence according to one embodiment.

FIG. 5 is a flowchart illustrating steps performed by the Uniquemer Engine 110 to generate a database of uniquemers according to one embodiment.

FIG. 6a tabulates results of applying the uniquemer algorithm to a set of protein sequences representing the proteome of Yersinia pestis. FIG. 6b tabulates the set of uniquemers identified in the protein sequence of putative F1 capsule anchoring protein, caf1A of Yersinia pestis.

FIGS. 7a and 7b illustrate the identified uniquemers relative to the three-dimensional protein structure of caf1A. FIG. 7a illustrates the display of the uniquemers on a front view of the caf1A protein structure. FIG. 7b illustrates the display of the uniquemers on a back view of the caf1A protein structure.

FIG. 8a tabulates results of applying the uniquemer algorithm to a set of protein sequences representing the proteome of the India 1967 strain of Variola virus. FIG. 8b tabulates the uniquemers found in the D13L protein sequence of the India 1967 of Variola virus.

FIGS. 9a and 9b illustrate the identified uniquemers relative to the three-dimensional protein structure of D13L. FIG. 9a illustrates the display of the uniquemers on a front view of the D13L protein structure. FIG. 9b illustrates the display of the uniquemers on a back view of the D13L protein structure.

The figures depict various embodiments of the present invention for purposes of illustration only. One skilled in the art will readily recognize from the following discussion that alternative embodiments of the illustrated structures and methods may be employed without departing from the principles of the described invention.

### DEFINITIONS

Residue: An amino acid residue is one amino acid that is joined to another by a peptide bond. Residue encompasses the combination of an amino acid and its position in a polypeptide sequence, for example, D31 or A234.

Surface residue: A surface residue is a residue located on a surface of a polypeptide. A surface residue usually includes a hydrophilic side chain. Operationally, a surface residue can be identified computationally from a structural model of a polypeptide as a residue that contacts a sphere of hydration rolled over the surface of the molecular structure. A surface residue also can be identified experimentally through the use of deuterium exchange studies, or accessibility to various labeling reagents such as, e.g., hydrophilic alkylating agents.

Buried residue: A buried residue is a residue that is not located on the surface of a polypeptide. Buried residues usually include a hydrophobic side chain.

Organism: A species, a strain of a species and a set of species (e.g., a genus or taxa).

Proteome: A set of protein sequences encoded by the genetic material (i.e., Ribonucleic Acid or Deoxyribose Nucleic Acid) of an organism. The proteome may contain all known protein sequences for an organism or a representative set of protein sequences for the organism.

Polypeptide: A single linear chain of 2 or more amino acids. A protein is an example of a polypeptide.

N-mer: A polypeptide of length n.

Uniquemer: A n-mer that is a sub-sequence of only one protein sequence (i.e., unique to a protein sequence) or an n-mer that is a sub-sequence of a set of protein sequences associated with only one organism (i.e., unique to an organism), a specified group of organisms (e.g., a genus), or a set of homologous protein sequences from different organisms (e.g., Stx1 shiga toxin).

Homolog: A gene related to a second gene by descent from a common ancestral DNA sequence. The term, homolog, may apply to the relationship between genes separated by the event of speciation or to the relationship between genes separated by the event of genetic duplication.

Taxonomy: The classification of organisms in an ordered system that indicates natural relationships. As discussed herein, taxonomy is a classification of organisms that indicates evolutionary relationships.

Conservation: Conservation is a high degree of similarity in the primary or secondary structure of molecules between homologs. This similarity is thought to confer functional importance to a conserved region of the molecule. In reference to an individual residue or amino acid, conservation is used to refer to a computed likelihood of substitution or deletion based on comparison with homologous molecules.

### DETAILED DESCRIPTION

The practice of the present invention will employ, unless otherwise indicated, conventional techniques of computational biology, biophysics, structural biology, evolutionary biology, molecular biology and biochemistry, which are within the skill of the art. Such techniques are explained fully in the literature, such as Singleton et al., Dictionary of Microbiology and Molecular Biology 2nd ed., J. Wiley & Sons (1994), Bourne et al., Structural Bioinformatics, J. Wiley & Sons (2002), Fogel et al., Evolutionary Computation in Bioinformatics, Morgan Kaufmann (2002) and Mount, Bioinformatics Sequence and Genome Analysis, Cold Spring Harbor Laboratory (2001).

As noted above, there is demand for a robust method of computationally determining protein signatures which provide the specific identification of an organism. Accordingly, the present invention provides a method for identifying protein subsequences that are unique to a protein sequence or an organism, i.e., ‘uniquemers,’ for development of detection assays and therapeutics.

These methods are widely applicable for identification of uniquemers representative of regions suitable for development of diagnostic reagents for proteins expressed by pathogens or associated with disease, virulence, or toxicity, or for development of therapeutic drugs or antibodies. Among the advantages provided by the present invention over prior art methods are reductions in the time and cost associated with such efforts by computational identification of protein regions that are optimal for reagent targeting. The identified regions are specific for a protein of interest or an organism encoding a set of proteins of interest. Consequently, reagents targeted to the identified regions are highly unlikely to cross-react with non-targeted regions. In addition, because all uniquemer subsequences of at least a specified length n are also uniquemers. Therefore, if only a subsequence of the identified uniquemer is responsible for binding to reagents, reagent specificity for the organisms of interest is not compromised. In other words, although the set of residues responsible for binding the reagent may be a subset of the uniquemer, cross-reaction with non-targeted residues or organisms will be minimized as each subsequence of length n within the uniquemer will also be unique.

The residues comprising identified uniquemer can be projected onto a three-dimensional protein structure corresponding to the protein sequence that includes the uniquemer to assist in evaluating the suitability of uniquemer residues for reagent development or bio-threat detection. Such methods provide a way to identify uniquemer residues on proteins that are surface exposed and amenable to binding by small molecule ligands or antibodies that will specifically recognize the reference polypeptide. Accordingly, the identification of uniquemers for an organism according to the methods of the invention enables development of reagents such as small chemical ligands or antibodies and assays using such reagents for highly specific detection of the target.

While the present method finds use in identifying uniquemers in any pathogen or target, preferred pathogens include but are not limited to, avian influenza, Ebola virus, dengue virus and the like. Others include SARS (coronavirus). Additionally, the same methods may be used for the detection of bacterial pathogens such as Bacillus anthracis, Escherichia coil, and Yersinia pestis. The method finds further use in the detection of plant based-toxins such as abrin and ricin.

In the development of therapeutics, the present invention finds use in the identification of uniquemers specific to a protein sequence or an organism. Identified uniquemers that provide special properties to the protein sequence or organism such as those conferring virulence, drug resistance or metastatic properties can be used to develop reagents to selectively block functionality of the regions.

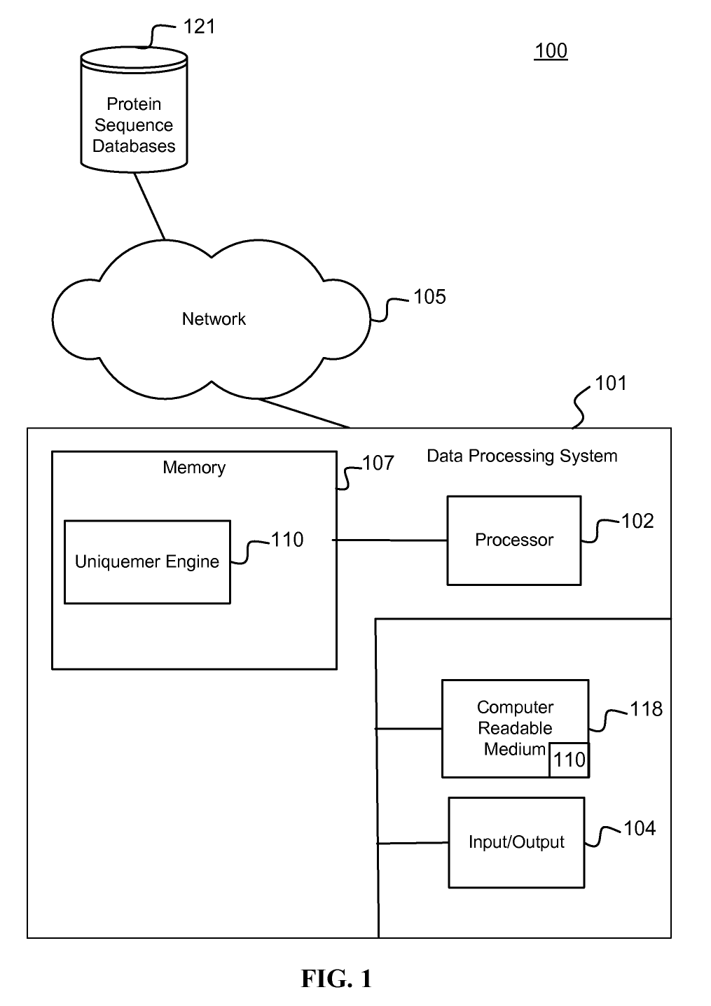

FIG. 1 shows a system architecture 100 adapted to support one embodiment of the present invention. FIG. 1 shows components used to identify protein subsequences that are unique to a given organism or protein sequence, i.e., “uniquemers.” The system architecture 100 includes a network 105, through which any number of Protein Sequence Databases 121 are accessed by a data processing system 101.

FIG. 1 shows component engines used to identify uniquemers. The data processing system 101 includes a Uniquemer Engine 110. The Uniquemer Engine 110 is implemented, in one embodiment, as software modules (or programs) executed by processor 102.

The Uniquemer Engine 110 operates to identify uniquemers based on protein sequences accessed from the Protein Sequence Databases 121 through the network 105 (as operationally and programmatically defined within the data processing system). According to the embodiment, Protein Sequence Databases 121 may include the Non Redundant set of protein sequence (NR) (available at the website of the National Institute for Bioinformatics Information) and SwissProt (available at the website of the European Bioinformatics Institute). Other Protein Sequence Databases 121 will be known to those skilled in the art.

It should also be appreciated that in practice at least some of the components of the data processing system 101 will be distributed over multiple computers, communicating over a network. For example, the Uniquemer Engine 110 may be deployed over multiple servers. As another example, the Uniquemer Engine 110 may be located on any number of different computers. For convenience of explanation, however, the components of the data processing system 101 are discussed as though they were implemented on a single computer.

In another embodiment, some or all of the Protein Sequence Databases 121 are located on the data processing system 101 instead of being coupled to the data processing system 101 by a network 105. For example, the Uniquemer Engine 110 may import protein sequences from Protein Sequence Databases 121 that are a part of or associated with the data processing system 101.

FIG. 1 shows that the data processing system 101 includes a memory 107 and one or more processors 102. The memory 107 includes the Uniquemer Engine 110. The Uniquemer Engine 110 is preferably implemented as instructions stored in memory 107 and executable by the processor 102.

In some embodiments, FIG. 1 also includes a computer readable medium 118 containing the Uniquemer Engine 110. FIG. 1 also includes one or more input/output devices 104 that allow data to be input and output to and from the data processing system 101. It will be understood that embodiments of the data processing system 101 also include standard software components such as operating systems and the like and further include standard hardware components not shown in the figure for clarity of example.

### Uniquemer Algorithm

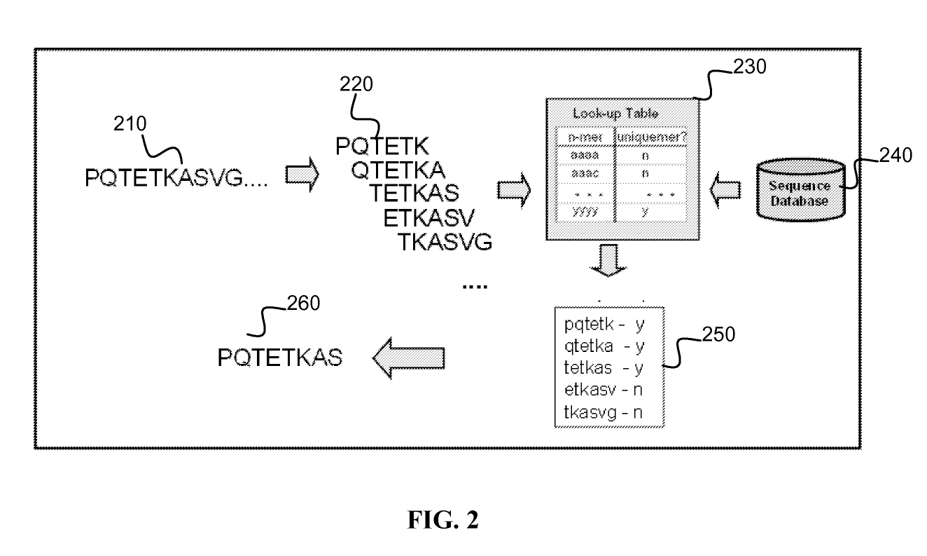

FIG. 2 provides a conceptual illustration of the Uniquemer algorithm, according to one embodiment. A protein sequence 210 is identified. A set of subsequences 220 are generated based on the protein sequence 210. A look-up table of uniquemers 230 is generated based on a protein sequence database 240 where each uniquemer in look-up table 230 only occurs in the database either once (i.e., in one protein sequence) or only in a set of protein sequences from a given organism. The generated set of subsequences is compared with the uniquemers in the lookup table to identify a subset of the subsequences that are uniquemers 250. This subset of subsequences is compared to the original protein sequence to identify uniquemers 260 in the protein sequences where all the subsequences of the uniquemer are also uniquemers.

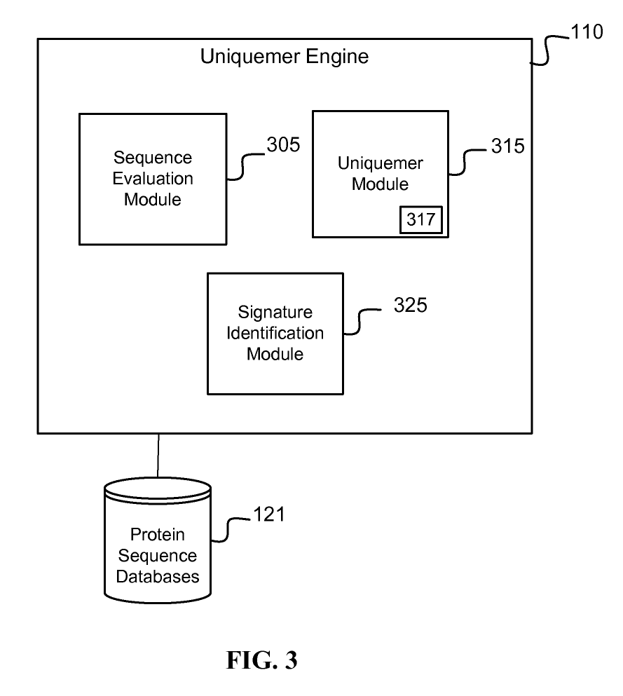

FIG. 3 illustrates a detailed view of the Uniquemer Engine 110 according to one embodiment. As shown in FIG. 3, the Uniquemer Engine 110 includes several modules. Those of skill in the art will recognize that other embodiments of the Uniquemer Engine 110 can have different and/or other modules than the ones described here, and that the functionalities can be distributed among the modules in a different manner.

The Uniquemer Engine 110 contains a Sequence Evaluation Module 305, a Uniquemer Module 315 and a Signature Identification Module 325. The Sequence Evaluation Module 305 functions to evaluate a set of one or more protein sequences to identify uniquemers in the one or more protein sequences. The Sequence Evaluation Module 305 is adapted to receive a set of protein sequences provided by a user, for example, using the one or more input/output devices 104. The Sequence Evaluation Module 305 is further adapted to retrieve protein sequences from the Protein Sequence Databases 121 using unique sequence identifiers provided by a user.

The Sequence Evaluation Module 305 generates a set of subsequences from the set of protein sequences. This set of subsequences can contain subsequences of different lengths. However, the majority of discussion of the present invention is directed to embodiments in which the set of subsequences in the set of protein sequences are of the same fixed length. These of subsequences are herein referred to as n-mers, where n represents the number of residues in the subsequence. Depending on the application of the present experiment, the n-mers can range from 4-10 residues in length. The n-mers preferably are 4, 5 or 6 residues in length.

In one embodiment, the Sequence Evaluation Module 305 generates the set of n-mers using a sliding window approach. In a sliding window approach, an n-mer of a fixed size is advanced one position in sequence to generate a set of n-mers, each n-mer differing from another by one residue.

The Sequence Evaluation Module 305 evaluates the set of generated n-mers to identify which n-mers are uniquemers using a lookup table 317 generated by the Uniquemer Module 315. The Sequence Evaluation Module 305 further identifies all uniquemers of size greater than n, where n is equal to the size of the generated n-mers. The Sequence Evaluation Module 305 identifies all uniquemers of size greater than n by identifying the start positions of the identified uniquemers. The start position of an identified uniquemer indicates a position of the first residues of the uniquemer in the protein sequence the uniquemer is a subsequence of. The Sequence Evaluation Module 305 determines a set of uniquemers that have start positions that differ by one residue. The Sequence Evaluation Module 305 then combines this set of uniquemers to generate a uniquemer of length greater than n.

The Uniquemer Module 315 is adapted to communicate with the Protein Sequence Databases 121 to identify a set of non-redundant protein sequences that represent known protein sequences. The Uniquemer Module 315 generates a lookup table 317 of uniquemers by identifying occurrence frequencies for a set of subsequences in the Protein Sequence Databases 121. An occurrence frequency indicates the number of times a subsequence occurs in the set of protein sequences specified in the Protein Sequence Databases 121. The Uniquemer Module 315 identifies subsequences in the Protein Sequence Databases 121 that have an occurrence frequency of one (i.e., are unique to a given sequence) or only occur in protein sequences associated with an organism (i.e., are unique to an organism) as uniquemers. In a specific embodiment, the Uniquemer Module 315 generates occurrence frequencies using a suffix tree algorithm. Another suitable method of generating occurrence frequencies for a set of subsequences in a dataset of sequences comprises using a sliding window approach over the entire dataset of sequences to identify subsequences, generating a hash or dictionary with each identified subsequence as a key and increasing the count by one each time that n-mer is encountered, storing it as the hash value for that key. Occurrence frequencies may also be generated by generating a set of all possible n-mers and using a regular expression or other similarity search method to ascertain the frequencies of each n-mer. The Uniquemer Module 315 stores the uniquemers subsequences in a lookup table 317. In a specific embodiment, the Uniquemer Module 315 stores all possible subsequences of a specified length in the lookup table in association with an indicator which specifies whether or not they are uniquemers.

The Signature Identification Module 325 provides visualizations of the uniquemer relative to three-dimensional protein structures and protein sequences to assist in the identification of uniquemers which form protein signatures. The Signature Identification Module 325 displays uniquemers onto a visualization of a three-dimensional protein structure of a protein sequence containing the uniquemer. Suitable methods for generating three-dimensional protein structures of a protein sequence are discussed in detail below in the section entitled Protein Structure Modeling.

The display of the uniquemers on the three-dimensional protein structures allows for the identification of a set of uniquemers residues on the surface of the protein that are proximate in three-dimensional space. This display is used to identify a set of residues that can be used for reagent development. This set can contain any number of residues but in most embodiments will be three or more residues, such as, e.g., three, four, five, or more residues. In alternate embodiments, the Signature Identification Module 325 identifies uniquemer residues that are proximate in three-dimensional space computationally, as described below in the Protein Structure Modeling section.

According to the application of the present invention, the Signature Identification Module 325 can use various programs for rendering the three-dimensional protein structure from a set of atom coordinates. RasMol is a common program for molecular graphics visualization. Other programs used to visualize three-dimensional protein structures include Chime and Protein Explorer. Such programs are well known to those of ordinary skill in the art.

The Signature Identification Module 325 further functions to project the uniquemers onto a linear representation of the protein sequence containing the uniquemer to visualize signatures of uniquemers contiguous in linear protein sequence. In one embodiment, the

Signature Identification Module 325 displays the uniquemers as colored residues in a visual representation of the protein sequences such as alphanumeric representation of the protein sequence or a line graph.

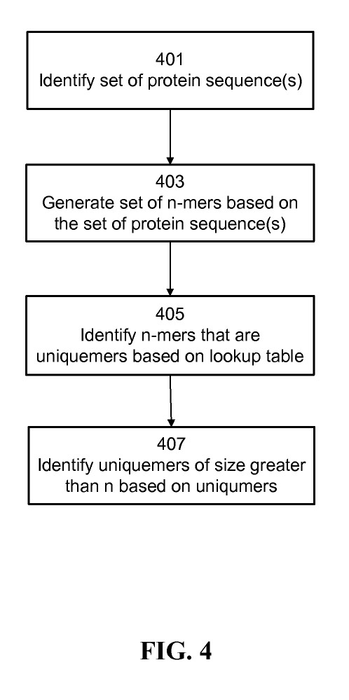

FIG. 4 is a flowchart illustrating steps performed by the Sequence Evaluation Module 305 to identify uniquemers in a set of one or more protein sequences according to one embodiment. Other embodiments perform the illustrated steps in different orders, and/or perform different or additional steps. Moreover, some of the steps can be performed by engines or modules other than the Sequence Evaluation Module 305.

The Sequence Evaluation Module 305 identifies 401 a set of protein sequences. The Sequence Evaluation Module 305 generates 403 a set of n-mers of based on the set of protein sequences. The Sequence Evaluation Module 305 identifies 405 which n-mers are uniquemers based on the lookup table 317 generated by the Uniquemer Module 315. The Sequence Evaluation Module 305 identifies 407 all uniquemers of size greater than n based on the identified uniquemers, where n is equal to the size of the generated n-mers.

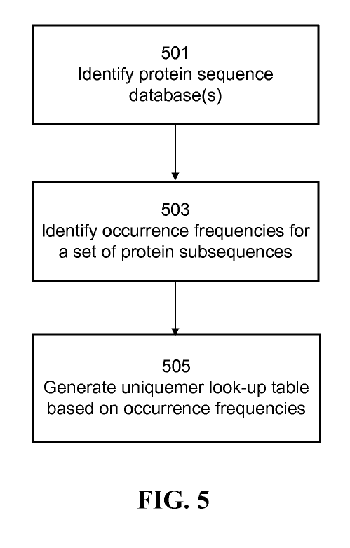

FIG. 5 is a flowchart illustrating steps performed by the Uniquemer Module 315 to generate a lookup table of uniquemers based on Protein Sequence Databases 121, according to one embodiment. Other embodiments perform the illustrated steps in different orders, and/or perform different or additional steps. Moreover, some of the steps can be performed by engines or modules other than the Uniquemer Module 315.

The Uniquemer Module 315 identifies 501 one or more Protein Sequence Database(s) 121. The Uniquemer Module 315 identifies 503 occurrence frequencies for a set of all n-mers based on the Protein Sequence Database(s) 121. The Uniquemer Module 315 generates 505 a lookup table 317 of uniquemers based on the occurrence frequencies and stores the uniquemers in a lookup table 317.

### Protein Structure Modeling

The protein structure used to display the uniquemer residues may be determined in a variety of methods. Protein structures are sets of solved atomic coordinates representative of a three dimensional structure of a protein. These coordinates are solved for atoms including, but not limited to, alpha carbons, beta carbons, or side chain atoms. These sets of solved atom coordinates can also represent some substructure of a protein or polypeptide. Atomic coordinates can be solved experimentally using a variety of techniques such as x-ray crystallography, electron crystallography and nuclear magnetic resonance.

Despite the accuracy of experimental techniques, they are costly and time-consuming. Advances in protein structure prediction or modeling provide methods of computationally solving the set of atom coordinates for a given protein. These methods are generally based on three different techniques (sequence comparison, threading and ab initio modeling). Protein structure prediction or modeling is usually practiced as a combination of these techniques.

A favored method in the art of protein structure prediction is to find a close homolog for whom the structure is known. CASP (Critical Assessment of Techniques for Protein Structure Prediction) (Moult et al., 2003) experiments have shown that protein structure prediction methods based on homology search techniques are still the most reliable prediction methods. Sequence comparison and threading techniques are based on homology search.

Sequence comparison approaches to protein structure prediction are popular due to availability of protein sequence information. These techniques use conventional sequence search and alignment techniques such as BLAST or FASTA to assign protein fold to the query sequence based on sequence similarity.

Approaches which use protein profiles are similar to sequence-sequence comparisons. A protein profile is an n-by-20 substitution matrix where n is the number of residues for a given protein. The substitution matrix is calculated via a multiple sequence alignment of close homologs of the protein. These profiles may be searched directly against sequence or compared with each other using search and alignment techniques such as PSI-BLAST and HMMer.

It is known that sequence similarity is not necessary for structural similarity. Proteins sharing similar structure can have negligible sequence similarity. Convergent evolution can drive completely unrelated proteins to adopt the same fold. Accordingly, ‘threading’ methods of protein structure prediction were developed which use sequence to structure alignments. In threading methods, the structural environment around a residue could be translated into substitution preferences by summing the contact preferences of surrounding amino acids. Knowing the structure of a template, the contact preferences for the 20 amino acids in each position can be calculated and expressed in the form of a n-by-20 matrix. This profile has the same format as the position specific scoring profile used by sequence alignment methods, such as PSI-BLAST, and can be used to evaluate the fitness of a sequence to a structure.

Ab initio methods are aimed at finding the native structure of the protein by simulating the biological process of protein folding. These methods perform iterative conformational changes and estimate the corresponding changes in energy. Ab initio methods are complicated by the inaccurate energy functions and the vast number of possible conformations a protein chain can adopt. The most successful approaches of ab initio modeling include lattice-based simulations of simplified protein models and methods building structures from fragments of proteins. Ab initio methods demand substantial computational resources and are also quite difficult to use and expert knowledge is needed to translate the results into biologically meaningful results. Despite known limitations, Ab initio methods are increasingly applied in large-scale annotation projects, including fold assignments for small genomes. Recent examples of such applications include: Bonneau et al. 2001, Kuhlman et al. 2003 and Dantas et al. 2003.

In practice, protein structure prediction typically involves a combination of the listed techniques, both experimental and computational. Hybrid approaches to protein structure prediction involve using different techniques for solving the atom coordinates at different stages or to solve for different parts of the protein structure. An example of this is the use of AS2TS (amino acid to tertiary structure, a homology modeling technique) to facilitate the molecular replacement (MR) phasing technique in experimental X-ray crystallographic determination of the protein structure of Mycobacterium tuberculosis (MTB) Rm1C epimerase (Rv3465) from the strain H37rv. The AS2TS system has been used to generate two homology models of this protein that were then successfully employed as MR targets.

Meta-predictors or consensus approaches attempt to benefit from the diversity of models by combining multiple techniques. In these methods, predictive models are collected and analyzed from a variety of different computational and experimental techniques. A common approach for combining models by consensus is to select the most abundant fold represented in the set of high scoring models. Other approaches to consensus modeling involve structural clustering such as HCPM-Hierarchical Clustering of Protein Models (Gront and Kolinski, 2005).

In one embodiment of the present invention the protein structures are predicted using the AS2TS program. The AS2TS system uses homology modeling to translate sequence-structure alignment data into atom coordinates. For a given sequence of amino acids, the AS2TS (amino acid sequence to tertiary structure) system calculates (e.g., using PSI-BLAST analysis of PDB) a list of the closest proteins from the PDB, and then a set of draft 3D models is automatically created.

### Example 1

### Identification of Uniquemers in Yersinia pestis

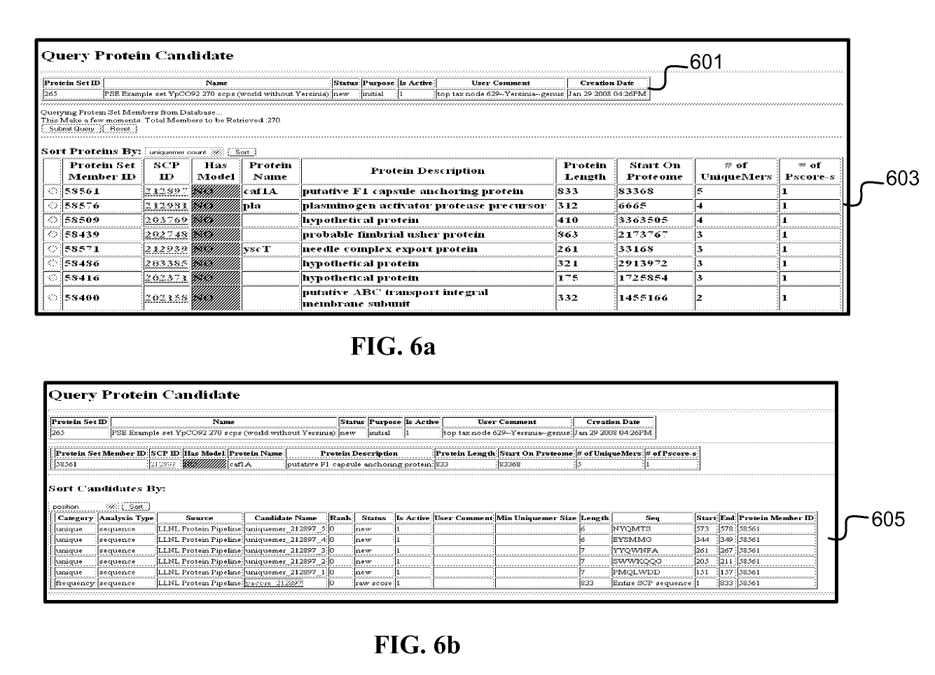

FIG. 6a illustrates tabulated results of applying the uniquemer algorithm to a set of protein sequences representing the proteome of Yersinia pestis. In this example, a query 601 was performed to select a set of protein sequences representing the proteome of Yersinia pestis. Uniquemer analysis was then applied to the set of protein sequences representing the proteome of Yersinia pestis to select a set of protein sequences containing uniquemers. The set of Yersinia pestis protein sequences containing uniquemers 603 was sorted according to the number of uniquemers identified in each protein sequence.

FIG. 6b illustrates the set of uniquemers 605 identified in the protein sequence of putative F1 capsule anchoring protein, caf1A of Yersinia pestis. The subsequences of the set of uniquemers identified in the protein sequence are shown in the column labeled ‘Seq’. The start and end positions of the uniquemer subsequences relative to the caf1A protein sequence are tabulated in the columns labeled ‘Start’ and ‘End’ respectively.

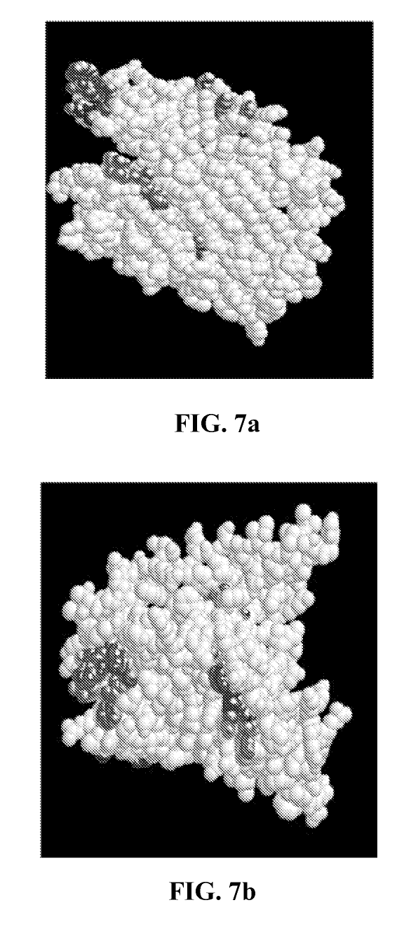

FIGS. 7a and 7b illustrate the identified uniquemers relative to the three-dimensional protein structure of caf1A. The protein structure of caf1A was modeled using the homology-based protein structure modeling system AS2TS (Zemla et al., 2005). The uniquemer residues tabulated in FIG. 6b are visualized on the surface of three-dimensional protein structure in gray. FIG. 7a illustrates the display of the uniquemers on a front view of the caf1A protein structure. FIG. 7b illustrates the display of the uniquemers on a back view of the caf1A protein structure. Visualization of surface-exposed regions containing residues with uniquemers was facilitated using RasMol (Sayle and Milner-White, 1995) to color uniquemer residues. Uniquemers were loaded into the b-factor column of the reference caf1A 3D coordinates file and displayed using RasMol's color-temperature setting.

### Example 2

### Identification of Signatures in the West Nile Virus Envelope Glycoprotein

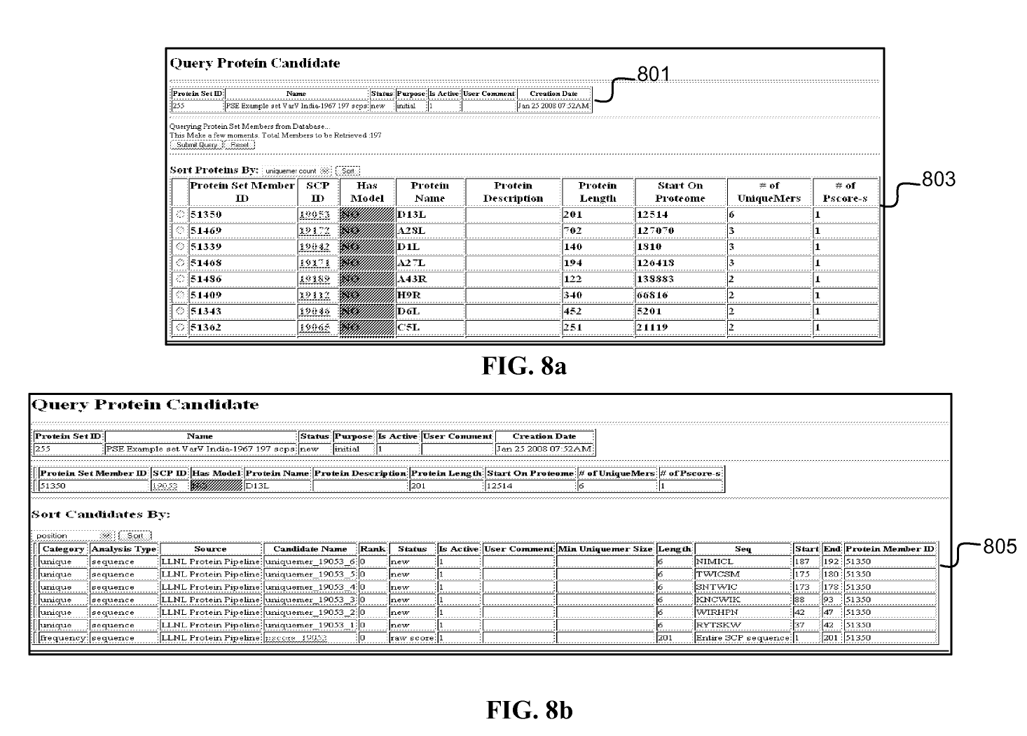

FIG. 8a illustrates tabulated results of applying the uniquemer algorithm to a set of protein sequences representing the proteome of the India 1967 strain of Variola virus (“Variola India 1967”). In this example, a query 801 was made to select a set of protein sequences representing the proteome of Variola India 1967. Uniquemer analysis was then applied to the set of protein sequences representing the proteome of Variola India 1967 to select a set of protein sequences containing uniquemers 803. The set of protein sequences containing uniquemers 802 was sorted according to the number of uniquemers identified in each protein sequence.

FIG. 8b illustrates the set of uniquemers 805 identified in the D13L protein sequence of Variola India 1967. The uniquemers identified in the sequence are shown in the column labeled ‘Seq’. The start and end positions of the uniquemer subsequences relative to the D13L protein sequence are tabulated in the columns labeled ‘Start’ and ‘End’ respectively.

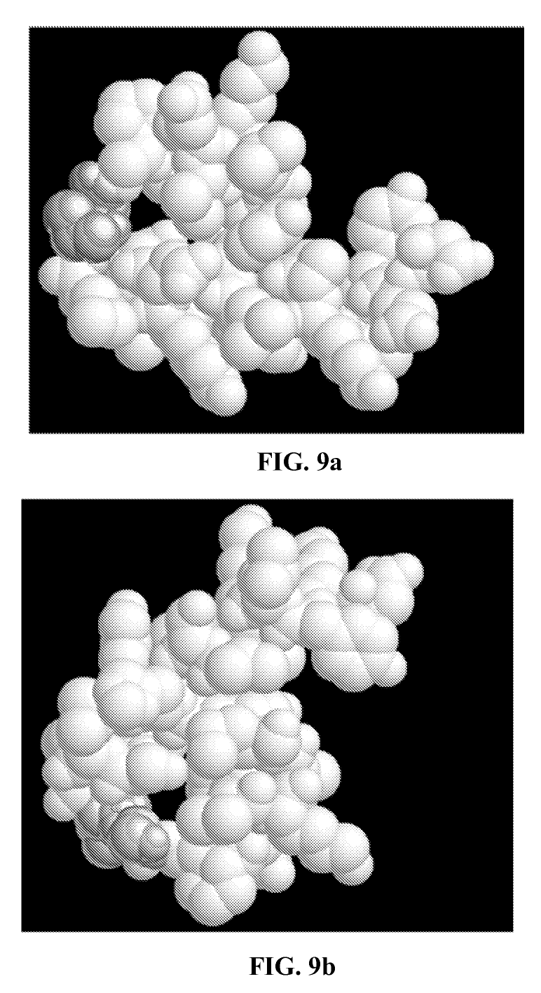

FIGS. 9a and 9b illustrate the identified uniquemers relative to the three-dimensional protein structure of D13L. The protein structure of D13L was modeled using the homology-based protein structure modeling system AS2TS (Zemla et al., 2005). The uniquemer residues are visualized on the surface of three-dimensional protein structure in gray. FIG. 9a illustrates the display of the uniquemers on a front view of the D13L protein structure. FIG. 9b illustrates the display of the uniquemers on a back view of the D13L protein structure. Visualization of surface-exposed regions containing residues with uniquemers was facilitated using RasMol (Sayle and Milner-White, 1995) to color uniquemer residues. Uniquemers were loaded into the b-factor column of the reference D13L 3D coordinates file and displayed using RasMol's color-temperature setting.

The description of the embodiments of the invention has been presented for the purpose of illustration; it is not intended to be exhaustive or to limit the invention to the precise forms disclosed. Persons skilled in the relevant art can appreciate that many modifications and variations are possible in light of the above teachings.

Some portions of above description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

In addition, the terms used to describe various quantities, data values, and computations are understood to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or the like, refer to the action and processes of a computer system or similar electronic computing device, which manipulates and transforms data represented as physical (electronic) quantities within the computer system memories or registers or other such information storage, transmission, or display devices.

Embodiments of the invention may also relate to an apparatus for performing the operations herein. This apparatus may be specially constructed for the required purposes, or it may comprise a general-purpose computing device selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

Embodiments of the invention may also relate to a computer data signal embodied in a carrier wave, where the computer data signal includes any embodiment of a computer program product or other data combination described herein. The computer data signal is a product that is presented in a tangible medium and modulated or otherwise encoded in a carrier wave transmitted according to any suitable transmission method.

The algorithms and displays presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will appear from the description above. In addition, embodiments of the invention are not described with reference to any particular programming language. It is appreciated that a variety of programming languages may be used to implement various embodiments of the invention as described herein, and any references to specific languages are provided for disclosure of enablement and best mode of embodiments of the invention.

All references disclosed in this specification, including references to books, scientific articles, patent applications, patents, and other publications are hereby incorporated by reference in their entirety for all purposes.

### REFERENCES

Moult, J., Fidelis, K., Zemla, A. (2003) Hubbard T., Critical assessment of methods of protein structure prediction (CASP)-round V., Proteins.; 53 Suppl 6:334-9.
Prager, E. M., Wilson, A. C. (1978) Construction of phylogenetic trees for proteins and nucleic acids: empirical evaluation of alternative matrix methods. J Mol Evol. June 20; 11(2):129-42.
Bonneau, R., Tsai, J., Ruczinski, I. and Baker, D. (2001) Functional inferences from blind ab initio protein structure predictions. J. Struct. Biol., 134, 186-190.
Kuhlman, B., Dantas, G., Ireton, G. C., Varani, G., Stoddard, B. L. and Baker, D. (2003) Design of a novel globular protein fold with atomic-level accuracy. Science, 302, 1364-1368. 61.
Dantas, G., Kuhlman, B., Callender, D., Wong, M. and Baker, D. (2003) A large scale test of computational protein design: folding and stability of nine completely redesigned globular proteins. J. Mol. Biol., 332, 449-460.
Attwood, T. K., Avison, H., Beck, M. E., Bewley, M., Bleasby, A. J., Brewster, F., Cooper, P., Degtyarendko, K., Geddes, A. J., Flower, D. R., Kelly, M. P., Lott, S., Measures, K. M., Parry-Smith, D. J., Perkins, D. N., Scordis, P., Scott, D., and Worledge, C. (1997) The PRINTS database of protein fingerprints: A novel information resource for computational molecular biology. J Chem Inf Comput Sci, 37, 417-424.
Berman, H. M., Westbrook, J., Feng, Z., Gilliland, G., Bhat, T. N., Weissig, H., Shindyalov, I. N., and Bourne, P. E. (2000) The protein data bank. Nucleic Acids Research, 8, 235-242.
Bower, M. J., Cohen, F. E. and Dunbrack, R. L. (1997) Prediction of protein side-chain rotamers from a backbone-dependent rotamer library: a new homology modeling tool. J Mol Biol, 267, 1268-1282.
Canutescu A. A., Shelenkov A. A. and Dunbrack, R. L. (2003) A graph theory algorithm for protein side-chain prediction. Prot Sci, 12, 2001-2014.
Gardner, S., Lam, M. W., Mulakken, N. J., Torres, C. L., Smith, J. R. and Slezak, T. R. (2004) Sequencing needs for viral diagnostics. Journal of Clinical Microbiology, 42, 0095-1137.
Hubbard, S. J. and Thornton, J. M. (1993) ‘NACCESS’, Computer Program, Department of Biochemistry and Molecular Biology, University College, London.
Karlin, S. and Ghandour, G. (1985) Multiple-alphabet amino acid sequence comparison of the immunoglobulin k-chain constant domain. Proc. Natl. Acad. Sci. USA, 82, 8597-8601.
Sayle, R. A. and Milner-White, E. J. 1995. RasMol: Biomolecular graphics for all. Trends in Biochemical Sciences, 20, 374-376.
Shuker, S. B., Hajduk, P. J., Meadows, R. P. and Fesik, S. W. (1996) Discovering High-Affinity Ligands for Proteins: SAR by NMR. Science, 274, 1531-1534.
Slezak, T., Kuczmarski, T., Ott, L., Torres, C., Medeiros, D., Smith, J., Truitt, B., Mulakken, N., Lam, M., Vitalis, E., Zemla, A., Zhou, C. E. and Gardner, S. (2003) Comparative genomics tools applied to bioterrorism defense. Briefings in Bioinformatics, 4, 133-149.
Wang, G., De, J., Schoeniger, J. S., Roe, D. C. and Carbonell, R. G. (2004) A hexamer peptide ligand that binds selectively to staphylococcal enterotoxin B: isolation from a solid phase combinatorial library. Journal of Peptide Research, 64, 51-64.
Zemla, A. (2003) LGA: a method for finding 3D similarities in protein structures. Nucleic Acid Research, 31, 3370-3374.
Zemla, A., Ecale Zhou, C., Slezak, T., Kuczmarski, T., Rama, D., Torres, C, Sawicka, D. and Barsky, D. (2005) AS2TS system for protein structure modeling and analysis. Nucleic Acids Research, 1;33(Web Server issue):W111-5.

What is claimed is:

1. A computer-implemented method of identifying a sub-sequence that is unique to an organism, the method comprising:
identifying a first protein sequence associated with the organism, wherein the first protein sequence comprises a plurality of ordered residues;
generating a plurality of sub-sequences based on the first protein sequence, wherein each sub-sequence comprises a plurality of contiguous residues and a starting residue number of each sub-sequence differs from a starting residue number of another sub-sequence by one position in the first protein sequence;
identifying a first unique sub-sequence comprising a first set of contiguous residues based on the plurality of sub-sequences, wherein the first unique sub-sequence is specific to the organism and is identified based on a dataset of protein sequences; and
storing the first unique sub-sequence.

2. The method of claim 1, further comprising:
identifying a second unique sub-sequence comprising a second set of contiguous residues based on the plurality of sub-sequences, wherein a starting residue number of the first unique sub-sequence and a starting residue number of the second unique sub-sequence differ by one position in the protein sequence and wherein the second unique sub-sequence is specific to the organism and is identified based on the dataset of protein sequences.

3. The method of claim 2, further comprising:
assembling the first unique sub-sequence and the second unique sub-sequence to generate a third unique sub-sequence; and
storing the third unique sub-sequence.

4. The method of claim 3, wherein the first sub-sequence and second unique sub-sequence are of length n, and all sub-sequences of length n or more of the third unique sub-sequence are specific to the organism.

5. The method of claim 1, wherein said dataset of protein sequences is a non redundant set of known protein sequences.

6. The method of claim 1, wherein each unique sub-sequence is identified based on a single occurrence of a sub-sequence of the plurality of sub-sequences within a dataset of protein sequences.

7. The method of claim 6, wherein each unique sub-sequence is identified based on a plurality of occurrences of a sub-sequence within a dataset of protein sequences, wherein the plurality of occurrences of the sub-sequence are based on protein sequences associated with the organism.

8. The method of claim 1, wherein identifying a set of unique sub-sequences based on the plurality of sub-sequences comprises:
generating a table of unique sub-sequences based on the dataset of protein sequences; and
identifying the set of unique sub-sequences based on the table of unique sub-sequences.

9. The method of claim 8, wherein the table is generated using a suffix tree algorithm.

10. The method of claim 1, wherein each sub-sequence of the plurality of sub-sequences comprises at least 4 residues.

11. The method of claim 10, wherein each sub-sequence of the plurality of sub-sequences comprises at least 5 residues.

12. The method of claim 1, further comprising displaying the set of unique sub-sequences onto a representation of a three-dimensional structure of the first protein sequence.

13. A computer-implemented method of identifying a sub-sequence that is unique to an organism, the method comprising:
identifying a first protein sequence associated with the organism, wherein the first protein sequence comprises a plurality of ordered residues;
generating a plurality of sub-sequences based on the first protein sequence, wherein each sub-sequence comprises a plurality of contiguous residues ranging from 4-10 residues in length;
identifying a first unique sub-sequence comprising a first set of contiguous residues based on the plurality of sub-sequences, wherein the first unique sub-sequence is specific to the organism and is identified based on a dataset of protein sequences; and
storing the first unique sub-sequence.

14. The method of claim 12, wherein the plurality of contiguous residues ranges from 5-9 residues in length.

15. The method of claim 12, wherein the plurality of contiguous residues ranges from 6-8 residues in length.

16. A computer-readable storage medium encoded with executable program code for identifying a sub-sequence that is unique to an organism, the program code comprising program code for:
identifying a first protein sequence associated with the organism, wherein the first protein sequence comprises a plurality of ordered residues;
generating a plurality of sub-sequences based on the first protein sequence, wherein each sub-sequence comprises a plurality of contiguous residues and a starting residue number of each sub-sequence differs from a starting residue number of another sub-sequence by one position in the first protein sequence;
identifying a first unique sub-sequence comprising a first set of contiguous residues based on the plurality of sub-sequences, wherein the first unique sub-sequence is specific to the organism and is identified based on a dataset of protein sequences; and
storing the first unique sub-sequence.

17. The medium of claim 16, further comprising program code for:
identifying a second unique sub-sequence comprising a second set of contiguous residues based on the plurality of sub-sequences, wherein a starting residue number of the first unique sub-sequence and a starting residue number of the second unique sub-sequence differ by one position in the protein sequence and wherein the second unique sub-sequence is specific to the organism and is identified based on the dataset of protein sequences.

18. The medium of claim 17, further comprising program code for:
assembling the first unique sub-sequence and the second unique sub-sequence to generate a third unique sub-sequence; and
storing the third unique sub-sequence.

19. The medium of claim 18, wherein the first sub-sequence and second unique sub-sequence are of length n, and all sub-sequences of length n or more of the third unique sub-sequence are specific to the organism.

20. A computer-readable storage medium encoded with executable program code for identifying a sub-sequence that is unique to an organism, the program code comprising program code for:
identifying a first protein sequence associated with the organism, wherein the first protein sequence comprises a plurality of ordered residues;
generating a plurality of sub-sequences based on the first protein sequence, wherein each sub-sequence comprises a plurality of contiguous residues ranging from 4-10 residues in length;
identifying a first unique sub-sequence comprising a first set of contiguous residues based on the plurality of sub-sequences, wherein the first unique sub-sequence is specific to the organism and is identified based on a dataset of protein sequences; and
storing the first unique sub-sequence.
